(19) United States
(12) Patent Application Publication
(10) Pub. No.: US 2025/0103812 A1
(43) Pub. Date: Mar. 27, 2025

(54) VERIFYING COMPLEX SENTENCES WITH ARTIFICIAL INTELLIGENCE

(71) Applicant: NEC Laboratories America, Inc., Princeton, NJ (US)

(72) Inventor: Christopher Malon, Fort Lee, NJ (US)

(21) Appl. No.: 18/888,763

(22) Filed: Sep. 18, 2024

Related U.S. Application Data
(60) Provisional application No. 63/540,426, filed on Sep. 26, 2023.

(51) Int. Cl. G06F 40/289; G06F 40/40; G06N 3/08; G06N 5/045; G16H 15/00

(52) U.S. Cl. CPC G06F 40/289; G06F 40/40; G06N 3/08; G06N 5/045; G16H 15/00

(57) ABSTRACT

Systems and methods for verifying complex sentences with artificial intelligence. Claim sentences can be filtered with source texts using a confirmation threshold, an unsupported threshold, and entailment probabilities computed by a natural language inference (NLI) classifier to obtain initial verification pairs. A trained imagination model can generate entailment outputs by employing initial verification pairs. A trained generalization model can generate generalized outputs by generalizing entailment outputs. Missing evidence generalizations can be chosen from sampled generalized outputs based on overlaps between sampled generalized outputs The NLI classifier can compute a final verification decision of the source texts against the missing evidence generalizations to obtain verified claim sentences. A corrective action for a monitored entity can be performed using the verified claim sentences.

### RELATED APPLICATION INFORMATION

This application claims priority to U.S. Provisional Application No. 63/540,426, filed on Sep. 26, 2023, incorporated herein by reference in its entirety.

### BACKGROUND

### Technical Field

The present invention relates to natural language processing with artificial intelligence and more particularly verifying complex sentences with artificial intelligence.

### Description of the Related Art

Artificial intelligence (AI) text generation models have improved dramatically. Texts generated by these models have become more human-like. However, concerns regarding the accuracy of the claims of AI-generated text, even human-generated text, still remain a persistent problem in natural language processing.

### SUMMARY

According to an aspect of the present invention, a computer-implemented method is provided for verifying complex sentences with artificial intelligence including, filtering claim sentences with source texts using a confirmation threshold, an unsupported threshold, and entailment probabilities computed by a natural language inference (NLI) classifier to obtain initial verification pairs, employing a trained imagination model to the initial verification pairs to generate entailment outputs, generalizing entailment outputs with a trained generalization model to generate generalized outputs, choosing missing evidence generalizations from sampled generalized outputs based on overlaps between sampled generalized outputs, computing a final verification decision of the source texts against the missing evidence generalizations using the NLI classifier to obtain verified claim sentences, and performing a corrective action for a monitored entity using the verified claim sentences.

According to another aspect of the present invention, a system is provided including, a memory device, one or more processor devices operatively coupled with the memory device to cause the processor device to: filter claim sentences with source texts using a confirmation threshold, an unsupported threshold, and entailment probabilities computed by a natural language inference (NLI) classifier to obtain initial verification pairs, employ a trained imagination model to the initial verification pairs to generate entailment outputs, generalize entailment outputs with a trained generalization model to generate generalized outputs, choose missing evidence generalizations from sampled generalized outputs based on overlaps between sampled generalized outputs, compute a final verification decision of the source texts against the missing evidence generalizations using the NLI classifier to obtain verified claim sentences, and perform a corrective action for a monitored entity using the verified claim sentences.

According to yet another aspect of the present invention, a non-transitory computer program product is provided including a computer-readable storage medium including program code for verifying complex sentences with artificial intelligence, wherein the program code when executed on a computer causes the computer to: filter claim sentences with source texts using a confirmation threshold, an unsupported threshold, and entailment probabilities computed by a natural language inference (NLI) classifier to obtain initial verification pairs, employ a trained imagination model to the initial verification pairs to generate entailment outputs, generalize entailment outputs with a trained generalization model to generate generalized outputs, choose missing evidence generalizations from sampled generalized outputs based on overlaps between sampled generalized outputs, compute a final verification decision of the source texts against the missing evidence generalizations using the NLI classifier to obtain verified claim sentences, and perform a corrective action for a monitored entity using the verified claim sentences.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram illustrating a high-level overview of a method for verifying complex sentences with artificial intelligence method, in accordance with an embodiment of the present invention;

FIG. 2 is a flow diagram illustrating a method for training an imagination model, in accordance with an embodiment of the present invention;

FIG. 3 is a flow diagram illustrating a method for training a generalization model, in accordance with an embodiment of the present invention;

FIG. 4 is a block diagram illustrating a system for verifying complex sentences with artificial intelligence, in accordance with an embodiment of the present invention;

FIG. 5 is a block diagram illustrating practical applications for a system for verifying complex sentences with artificial intelligence, in accordance with an embodiment of the present invention; and

FIG. 6 is a block diagram illustrating deep learning neural networks for verifying complex sentences with artificial intelligence, in accordance with an embodiment of the present invention.

### DETAILED DESCRIPTION OF PREFERRED EMBODIMENTS

In accordance with embodiments of the present invention, systems and methods are provided for verifying complex sentences with artificial intelligence.

In an embodiment, claims sentences can be filtered with source texts using a confirmation threshold, an unsupported threshold, and entailment probabilities computed by a natural language inference (NLI) classifier to obtain initial verification pairs. A trained imagination model can generate entailment outputs by employing initial verification pairs. A trained generalization model can generate generalized outputs by generalizing entailment outputs. Missing evidence generalizations can be chosen from sampled generalized outputs based on overlaps between sampled generalized outputs. The NLI classifier can compute a final verification decision of the source texts against the missing evidence generalizations to obtain verified claim sentences. A corrective action for a monitored entity can be performed using the verified claim sentences.

Many natural language processing (NLP) tasks involve the generation of output text based on provided input text or retrieved text (e.g., source text). Automatic factuality metrics score can be computed to determine whether a generated claim text is consistent with a source text. A generated text that is untrue or not consistent with sources may be called a “hallucination.”

A natural language inference (NLI) classifier can compare two texts (e.g., source text and claim sentences) to classify whether one implies or entails the other by analyzing semantic relationships, logical consequences, common knowledge, paraphrasing, context clues, presupposition, or grammatical structures of the texts. The NLI classifier can provide judgments of entailment probability which is the likelihood of whether each generated sentence (e.g., claim sentence) is supported by certain granularities (e.g., documents, paragraphs, sentence pairs, or sentences) of source text.

When support for a statement extends outside a lexical unit of an NLI classifier's level of granularity, the NLI classifier can fail to combine information spread throughout the source text in multiple places. By focusing on one locality of the source text, NLI classifiers estimate the probability that the outside context would support missing details not found in that location without actually verifying those details.

The present embodiments can perform a “multi-hop” fact checking method which extends sentence-level NLI comparisons with an imagination model and a generalization model, which together specify what information is lacking to complete the verification. The final score results from using multiple NLI classifications to verify the lacking information, which is simpler, against other parts of the source text.

The present embodiments improve accuracy of NLI classifiers particularly on entity, coreference, and mixed type hallucinations. The present embodiments also improve NLI classifiers by providing explanations of missing evidence for unverified claim sentences.

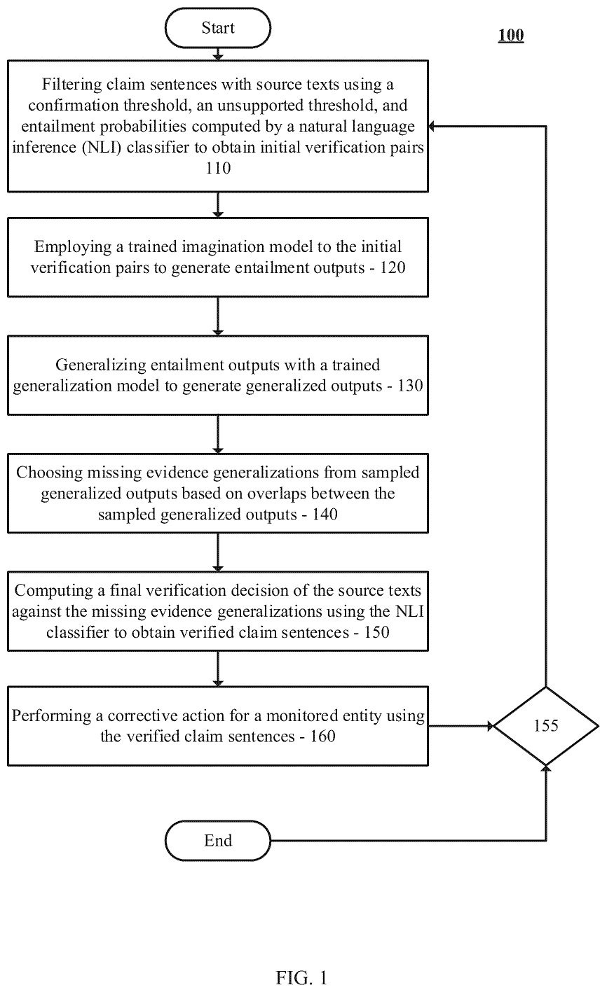

Referring now in detail to the figures in which like numerals represent the same or similar elements and initially to FIG. 1, a high-level overview of a method for verifying complex sentences with artificial intelligence is illustratively depicted in accordance with one embodiment of the present invention.

In an embodiment, claims sentences can be filtered with source texts using a confirmation threshold, an unsupported threshold, and entailment probabilities computed by a natural language inference (NLI) classifier to obtain initial verification pairs. A trained imagination model can generate entailment outputs by employing initial verification pairs. A trained generalization model can generate generalized outputs by generalizing entailment outputs. Missing evidence generalizations can be chosen by comparing overlaps between sampled generalized outputs. The NLI classifier can compute a final verification decision of the source texts against the missing evidence generalizations to obtain verified claim sentences. A corrective action for a monitored entity can be performed using the verified claim sentences.

Referring now to block 110 of FIG. 1, describing a method of filtering claim sentences with source texts using a confirmation threshold, an unsupported threshold, and entailment probabilities computed by a natural language inference (NLI) classifier to obtain initial verification pairs.

To filter the claim sentences with the source texts, the claim can be verified with the source texts using entailment probabilities. To verify a claim sentence, the NLI classifier computes an entailment probability between every sentence of the source text and the claim sentence. Source texts having the highest entailment probability with a claim sentence as “support” can form an initial verification pair with the claim sentence. The NLI classifier can save the highest entailment probability as the “initial probability.” The NLI classifier can be a bidirectional encoder representations from transformers (BERT) or text-to-text transfer transformer (T5). The NLI classifier can employ summarization consistency (SummaC). Other NLI classifiers can be utilized.

If the initial probability is above a confirmation threshold, the claim sentence is confirmed as supported and can be used to perform a corrective action as described further in block 160 of FIG. 1.

If the initial probability is below an unsupported threshold, which is lower than the confirmation threshold, the claim sentence is rejected as unsupported and the initial verification pair is discarded.

The confirmation threshold and unsupported threshold are hyperparameters between zero and one, and they may be set based on cross-validation or application requirements.

If the initial probability is between the two thresholds, the imagination module is applied to the initial verification pair that includes the claim sentence with the support as the (singleton) evidence list.

Referring now to block 120 of FIG. 1, describing an embodiment of a method of employing a trained imagination model to initial verification pairs to generate entailment outputs.

The imagination model can read text including the statement to be verified and partial support from the source documents, and outputs imagined outputs that includes the word “supports” or “refutes” with text representing what the remaining evidence that can support a claim sentence may look like, or the word “none” if the input evidence is already complete. A fact verification task can include a set of claims to be verified with labels “supported,” “refuted,” or “not enough information” (NEI), with a ground truth set of “gold” supporting sentences or table cells from a document corpus for each claim. The fact verification task can include classifying each claim and extracting the set of supporting evidences or table cells from the corpus. A total of k generations of the imagination module can be sampled, by using nucleus sampling. Entailment outputs are non-empty generations by the imagined model that came with an “entailment” label. The imagination model can employ a sequence-to-sequence transformer model, such as T5 or BART. Other sequence-to-sequence transformer model are contemplated.

If the number of imagined outputs having the word “refutes” are more than a refutes threshold, or the number of imagined outputs where the remaining evidence is not “none” is one or fewer, the claim sentence is rejected as unsupported. The refutes threshold can be a natural number based on the combination of the number of claim sentences and source texts such as three, twenty, etc.

Because of the training data of the imagination module, each imagined evidence generation is expected to hallucinate details beyond the details necessary to confirm the claim sentence. However, the present embodiments expect that each sampled imagined evidence generation hallucinates different details. If we can find a common sentence implied by all of the sampled imagined evidence generations, we expect that this commonality reflects what really still needs to be confirmed to verify the claim sentence.

Referring now to FIG. 2, describing an embodiment of a method of training the imagination model.

Block 210 of FIG. 2 describes an embodiment of a method of retrieving documents for claim texts.

A document retrieval method such as term frequence-inverse document frequency (TF-IDF). To perform the fact verification task, each input begins with the task identifier string such as “missing: ” and a list of the titles of pages retrieved already, followed by a claim identifier string such as “[HYP]”, and then the claim text being classified.

After an input separator, the elements of the current evidence set (each beginning with a page title in brackets) are concatenated. When applied to the summary verification task, where the source text may not have a page title, the first named entity of a sentence representing a person may be used as the title, or “News” if no such named entity is found.

Block 220 of FIG. 2 describes an embodiment of a method of constructing a training set from the documents in which unretrieved evidence is predicted from retrieved evidence and claim texts.

After retrieval, every example requiring evidence from an unretrieved document is used as an example, with the current evidence set being the gold evidence in the retrieved documents and the target evidence being the first piece of evidence from a missing document. For half of the remaining examples (those with no missing documents) including all NEI examples with multiple pieces of evidence, a piece of evidence is randomly left out from the current evidence set, and that evidence is to be predicted as the target. In the other examples, the word “none” is to be predicted, indicating that the evidence chain is complete.

Block 230 of FIG. 2 describes an embodiment of a method of fine-tuning a sequence-to-sequence neural network with the training set to predict target texts from source texts from the training set.

Training the imagination model can be based on the gold evidence chains in the training set, and the set of documents retrieved by the baseline model. A log likelihood objective function for training can be computed for the target output string as a multi-task objective, combining a prediction of missing evidence with a prediction of the label based on partial information.

The target texts can include the word “supports” or “refutes,” followed by the target evidence in the usual format which can include page title in brackets, followed by a sentence taken from that page, or “none.” For NEI examples, “supports” is to be predicted, indicating a partial evidence chain with no contradictions yet.

Referring back to FIG. 1 and pointing to block 130 describing an embodiment of a method of generalizing entailment outputs with a trained generalization model to generate generalized outputs.

Entailment outputs can be taken as input to the generalization module to generate generalized outputs. The generalization model can employ a sequence-to-sequence transformer model, such as T5 or BART. Other sequence-to-sequence transformer model are contemplated. The generalization module outputs generalized outputs which are sentences from claim sentences commonly implied by multiple input sentences (e.g., source texts).

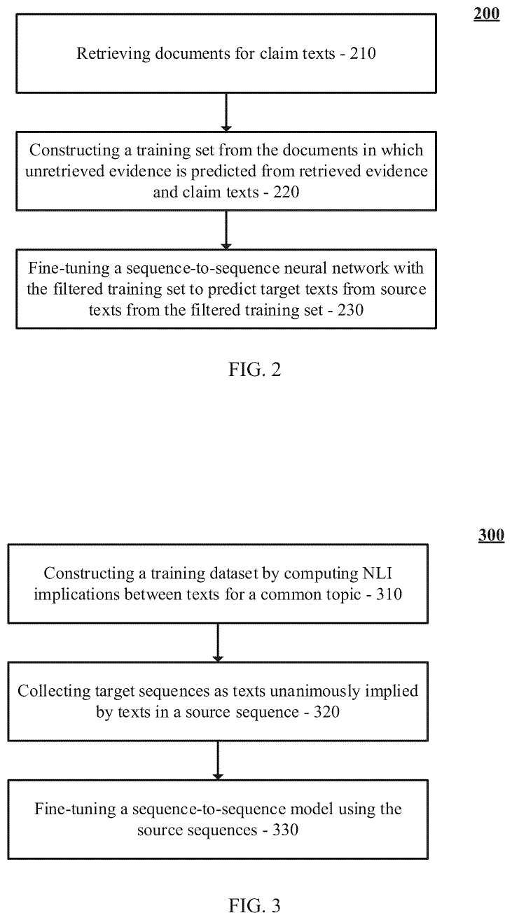

Referring now to FIG. 3, describing an embodiment of training the generalization model.

Block 310 of FIG. 3 describes an embodiment of a method where a training dataset for the generalization model can be constructed with support texts which are texts where the same conclusions regarding an entity are supported by different sentences. For example, the training dataset can include a multi-document summarization of eCommerce product reviews. The training data for the generalization model can be constructed by first running an NLI model to compute NLI implications (e.g., entailment, contradiction or neutral) on each pair of support texts sentences from distinct support texts for a common entity (e.g., a product) which can include a predetermined number (e.g., eight) of distinct support texts for a common entity. NLI implications can be computed with the SummaC model. The present embodiments define “weakly entailed” decisions to be decisions at a low entailment threshold, and “strongly entailed” decisions at a high entailment threshold.

The low entailment threshold and high entailment threshold are hyperparameters representing probability values between zero and one.

Block 320 of FIG. 3 describes an embodiment of a method of collecting target sequences as texts unanimously implied by texts in a source sequence.

Given the NLI implications, the present embodiments can sort the support texts in decreasing order by the number of other support texts that strongly entail them. The present embodiments can select support texts in order that are not weakly entailed by previously selected support texts, as long as each text in a source sequence is strongly entailed by at least one other support text. Each selected support text can become the target sequence in an example, where the source sequence is the set of support texts which strongly entail it, and concatenated with a vertical bar separator.

Block 330 of FIG. 3 describes an embodiment of a method of training the generalization model using cross-entropy loss to predict target sequences from source sequences.

Referring back to FIG. 1 and pointing to block 140 describing an embodiment of a method where missing evidence generalizations can be chosen from sampled generalized outputs by considering the token overlaps between the sampled generalized outputs.

Sampled generalized outputs can be generalization module outputs sampled using nucleus sampling. For each token of each sampled generalized output, the number of other sampled generalized outputs matching the token is counted, and the sampled generalized output with the highest average number of matching sampled generalized outputs per token can be selected as the best generalization. The best generalization can represent the missing evidence needed to support the claim sentence.

Block 150 of FIG. 1 describes an embodiment of a method of computing a final verification decision of the source texts against the missing evidence generalizations using the NLI classifier to obtain verified claim texts.

Verification of the claim sentence can continue by applying the NLI classifier between each sentence of the source text and the best generalization. The final verification decision as supported or unsupported can be determined by the highest entailment probability output by the NLI classifier for all source text sentences. In addition to the decision as supported or unsupported, source texts supporting the claim sentences (e.g., from the initial verification pair and from the from the source text sentence with the highest NLI score against the best generalization) can be outputted as an explanation for the decision. The explanation statements for unsupported decisions can include textual description of the unverified information as the missing evidence generalization.

In an embodiment as shown in block 155 of FIG. 1, the entire verification process of the present embodiments can be done iteratively to support the collection of at least two source texts supporting a claim sentence until an iteration threshold has been met. The iteration threshold can be a natural number such as three, twenty, etc.

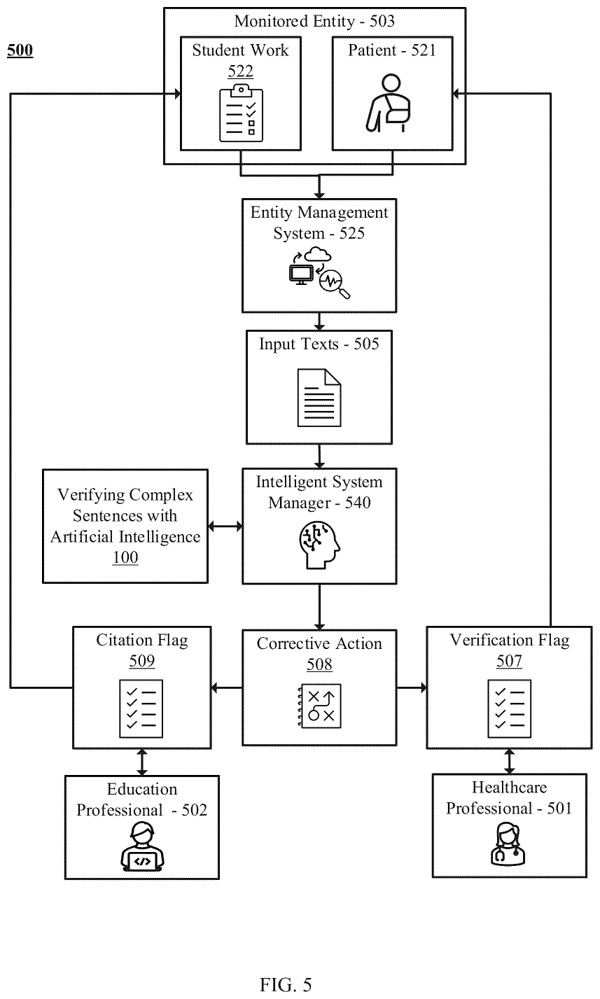

Block 160 of FIG. 1 describes an embodiment where a corrective action can be performed for a monitored entity using the verified claim sentences. This is described in more detail in FIG. 5.

Referring now to FIG. 5 which describes embodiments of practical applications for verifying complex sentences with artificial intelligence.

In an embodiment for a healthcare setting, a verification flag 507 (e.g., corrective action 508) can be generated by an intelligent system manager 540 for predicted healthcare summaries of healthcare data (e.g., input texts 505) for a patient 521 (e.g., monitored entity) obtained from a healthcare management system (e.g., entity management system 525) to assist the decision-making process of a healthcare professional. The verification flag 507 can represent whether the predicted healthcare summary is supported by the healthcare data. The intelligent system manager 540 can implement the computer-implemented method for verifying complex sentences with artificial intelligence 100. By using the verified claim sentences from the predicted healthcare summaries of healthcare data, a healthcare professional can update the medical diagnosis of the patient.

In an embodiment, the entity management system 525 and the intelligent system manager 540 can be implemented in different application systems that can be located in different geographical locations. In another embodiment, the entity management system 525 and the intelligent system manager 540 can be implemented in a single application system.

In another embodiment for the education field, a citation flag 509 (e.g., corrective action) can be generated for input texts 505 in student work 522 (e.g., monitored entity). The citation flag 509 can represent whether the input sentences are properly supported by cited materials. Other practical applications are contemplated for other fields such as legal, government, public service, etc.

The present embodiments improve accuracy of NLI classifiers by gaining significant accuracy metrics particularly on entity, coreference, and mixed type hallucinations. The present embodiments also improve NLI classifiers by providing explanations to missing evidence to verify claim sentences.

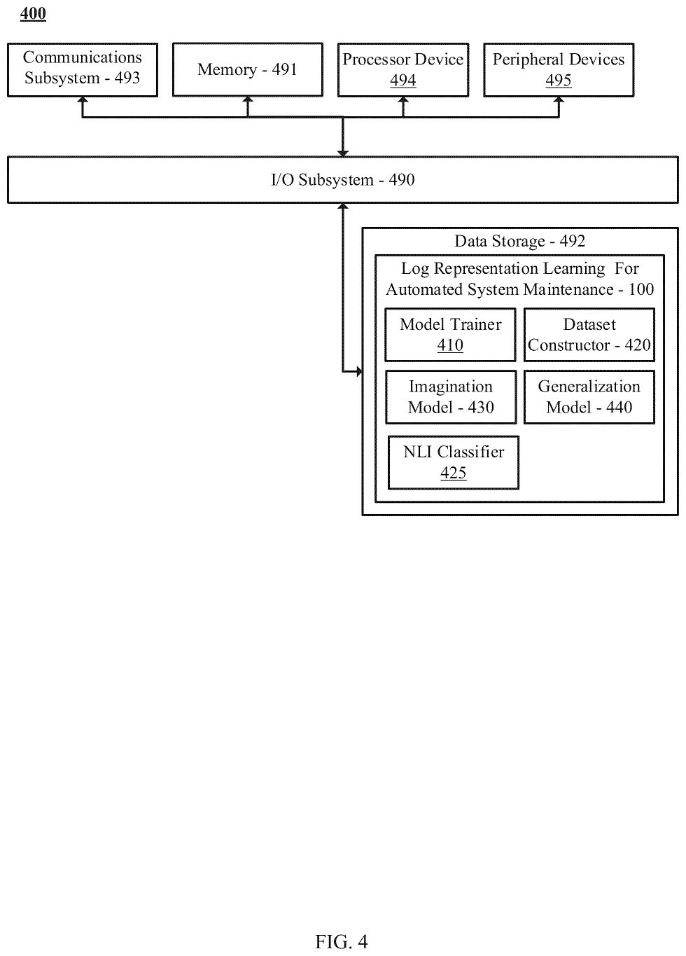

Referring now to FIG. 4, a system for verifying complex sentences with artificial intelligence is illustratively depicted in accordance with an embodiment of the present invention.

The computing device 400 illustratively includes the processor device 494, an input/output (I/O) subsystem 490, a memory 491, a data storage device 492, and a communication subsystem 493, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 491, or portions thereof, may be incorporated in the processor device 494 in some embodiments.

The processor device 494 may be embodied as any type of processor capable of performing the functions described herein. The processor device 494 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 491 may be embodied as any type of volatile or non-volatile memory or data storage capable of performing the functions described herein. In operation, the memory 491 may store various data and software employed during operation of the computing device 400, such as operating systems, applications, programs, libraries, and drivers. The memory 491 is communicatively coupled to the processor device 494 via the I/O subsystem 490, which may be embodied as circuitry and/or components to facilitate input/output operations with the processor device 494, the memory 491, and other components of the computing device 400. For example, the I/O subsystem 490 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 490 may form a portion of a system-on-a-chip (SOC) and be incorporated, along with the processor device 494, the memory 491, and other components of the computing device 400, on a single integrated circuit chip.

The data storage device 492 may be embodied as any type of device or devices configured for short-term or long-term storage of data such as, for example, memory devices and circuits, memory cards, hard disk drives, solid state drives, or other data storage devices. The data storage device 492 can store program code for verifying complex sentences with artificial intelligence 100. The program code for verifying complex sentences with artificial intelligence 100 can include a model trainer 410 that can train artificial intelligence models with datasets. The program code for verifying complex sentences with artificial intelligence 100 can include a dataset constructor 420 that can construct a training dataset from inputs provided such as documents, text, existing datasets. The program code for verifying complex sentences with artificial intelligence 100 can fine-tune an imagination model 430 to generate entailment outputs using a NLI Classifier 425 and a generalization model 440 to generate generalized outputs. Any or all of these program code blocks may be included in a given computing system.

The communication subsystem 493 of the computing device 400 may be embodied as any network interface controller or other communication circuit, device, or collection thereof, capable of enabling communications between the computing device 400 and other remote devices over a network. The communication subsystem 493 may be configured to employ any one or more communication technology (e.g., wired or wireless communications) and associated protocols (e.g., Ethernet, InfiniBand®, Bluetooth®, Wi-Fi®, WiMAX, etc.) to affect such communication.

As shown, the computing device 400 may also include one or more peripheral devices 495. The peripheral devices 495 may include any number of additional input/output devices, interface devices, and/or other peripheral devices. For example, in some embodiments, the peripheral devices 495 may include a display, touch screen, graphics circuitry, keyboard, mouse, speaker system, microphone, network interface, and/or other input/output devices, interface devices, GPS, camera, and/or other peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing system 400 are readily contemplated by one of ordinary skill in the art given the teachings of the present invention provided herein.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid-state memory, magnetic tape, a removable computer diskette, a random-access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

The present embodiments can employ deep learning neural networks such as the imagination model and the generalization model.

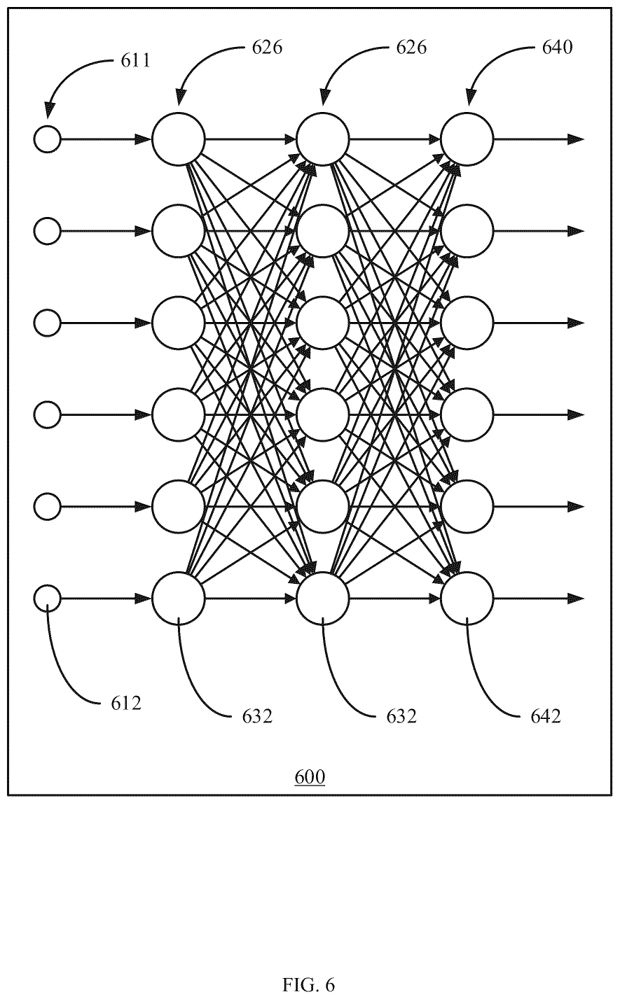

Referring now to FIG. 6, a block diagram illustrating deep learning neural networks for verifying complex sentences with artificial intelligence, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 600, such as a multilayer perceptron, can have an input layer 611 of source nodes 612, one or more computation layer(s) 626 having one or more computation nodes 632, and an output layer 640, where there is a single output node 642 for each possible category into which the input example can be classified. An input layer 611 can have a number of source nodes 612 equal to the number of data values 612 in the input data 611. The computation nodes 632 in the computation layer(s) 626 can also be referred to as hidden layers, because they are between the source nodes 612 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the inputted data. A deep neural network can be fully connected, where each node in a computational layer is connected to all other nodes in the previous layer, or may have other configurations of connections between layers. If links between nodes are missing, the network is referred to as partially connected.

In an embodiment, the computation layers 626 of the imagination model 430 used can learn the semantic relationships, logical consequences, common knowledge, paraphrasing, context clues, presupposition, or grammatical structures of at least two input texts. The output layer 640 of the imagination model 430 can then provide the overall response of the network as a likelihood score as an entailment probability. In an embodiment, the computation layers 626 of the generalization model 440 used can learn the semantic relationships, logical consequences, common knowledge, paraphrasing, context clues, presupposition, or grammatical structures of at least two input texts. The output layer 640 of the generalization model 440 can then provide the overall response of the network as a likelihood score as an NLI implication.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 626 perform a nonlinear transformation on the input data 612 that generates a feature space. The classes or categories may be more easily separated in the feature space than in the original data space.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for verifying complex sentences with artificial intelligence comprising:
filtering claim sentences with source texts using a confirmation threshold, an unsupported threshold, and entailment probabilities computed by a natural language inference (NLI) classifier to obtain initial verification pairs;
employing a trained imagination model to the initial verification pairs to generate entailment outputs;
generalizing entailment outputs with a trained generalization model to generate generalized outputs;
choosing missing evidence generalizations from sampled generalized outputs based on overlaps between the sampled generalized outputs;
computing a final verification decision of the source texts against the missing evidence generalizations using the NLI classifier to obtain verified claim sentences; and
performing a corrective action for a monitored entity using the verified claim sentences.

2. The computer-implemented method of claim 1, wherein the trained imagination model is obtained by:
retrieving documents for claim texts;
constructing a training set from the documents in which unretrieved evidence is predicted from retrieved evidence and claim texts; and
fine-tuning a sequence-to-sequence neural network with the training set to predict target texts from source texts from the training set.

3. The computer-implemented method of claim 1, wherein the trained generalization model is obtained by:
constructing a training dataset by computing NLI implications between texts for a common topic;
collecting target sequences as texts unanimously implied by texts in a source sequence; and
fine-tuning a sequence-to-sequence model using the source sequences and target sequences.

4. The computer-implemented method of claim 1, wherein performing the corrective action further comprises generating a verification flag for predicted healthcare summaries of healthcare data of a patient obtained from a healthcare management system to assist a decision-making process of a healthcare professional.

5. The computer-implemented method of claim 1, further comprising generating explanation statements for unsupported decisions that includes textual description of unverified information.

6. The computer-implemented method of claim 5, wherein the textual description is employed to find additional supporting sentences.

7. The computer-implemented method of claim 1, wherein the imagination model simultaneously predicts missing evidence and entailment outputs including classifications of “supports” and “refutes”.

8. A system comprising:
a memory device; and
one or more processor devices operatively coupled with the memory device to cause the processor device to:
filter claim sentences with source texts using a confirmation threshold, an unsupported threshold, and entailment probabilities computed by a natural language inference (NLI) classifier to obtain initial verification pairs;
employ a trained imagination model to the initial verification pairs to generate entailment outputs;
generalize entailment outputs with a trained generalization model to generate generalized outputs;
choose missing evidence generalizations from sampled generalized outputs based on overlaps between the sampled generalized outputs;
compute a final verification decision of the source texts against the missing evidence generalizations using the NLI classifier to obtain verified claim sentences; and
perform a corrective action for a monitored entity using the verified claim sentences.

9. The system of claim 8, wherein the trained imagination model is obtained by:
retrieving documents for claim texts;
constructing a training set from the documents in which unretrieved evidence is predicted from retrieved evidence and claim texts; and
fine-tuning a sequence-to-sequence neural network with the training set to predict target texts from source texts from the training set.

10. The system of claim 8, wherein the trained generalization model is obtained by:
constructing a training dataset by computing NLI implications between texts for a common topic;
collecting target sequences as texts unanimously implied by texts in a source sequence; and
fine-tuning a sequence-to-sequence model using the source sequences and target sequences.

11. The system of claim 8, wherein performing the corrective action further comprises generating a verification flag for predicted healthcare summaries of healthcare data obtained from a healthcare management system to assist a decision-making process of a healthcare professional.

12. The system of claim 8, further comprising generating explanation statements for unsupported decisions that includes textual description of unverified information.

13. The system of claim 12, wherein the textual description is employed to find additional supporting sentences.

14. The system of claim 8, wherein the imagination model simultaneously predicts missing evidence and entailment outputs including classifications of “supports” and “refutes”.

15. A non-transitory computer program product comprising a computer-readable storage medium including program code for verifying complex sentences with artificial intelligence, wherein the program code when executed on a computer causes the computer to:
filter claim sentences with source texts using a confirmation threshold, an unsupported threshold, and entailment probabilities computed by a natural language inference (NLI) classifier to obtain initial verification pairs;
employ a trained imagination model to the initial verification pairs to generate entailment outputs;
generalize entailment outputs with a trained generalization model to generate generalized outputs;
choose missing evidence generalizations from sampled generalized outputs based on overlaps between the sampled generalized outputs;
compute a final verification decision of the source texts against the missing evidence generalizations using the NLI classifier to obtain verified claim sentences; and
perform a corrective action for a monitored entity using the verified claim sentences.

16. The non-transitory computer program product of claim 15, wherein the trained imagination model is obtained by:
retrieving documents for claim texts;
constructing a training set from the documents in which unretrieved evidence is predicted from retrieved evidence and claim texts; and
fine-tuning a sequence-to-sequence neural network with the training set to predict target texts from source texts from the training set.

17. The non-transitory computer program product of claim 15, wherein the trained generalization model is obtained by:
constructing a training dataset by computing NLI implications between texts for a common topic;
collecting target sequences as texts unanimously implied by texts in a source sequence; and
fine-tuning a sequence-to-sequence model using the source sequences and target sequences.

18. The non-transitory computer program product of claim 15, wherein performing the corrective action further comprises generating a verification flag for predicted healthcare summaries of healthcare data obtained from a healthcare management system to assist a decision-making process of a healthcare professional.

19. The non-transitory computer program product of claim 15, further comprising generating explanation statements for unsupported decisions that includes textual description of unverified information to find additional supporting sentences.

20. The non-transitory computer program product of claim 15, wherein the imagination model simultaneously predicts missing evidence and entailment outputs including classifications of “supports” and “refutes”.
